(19) United States
(12) Patent Application Publication
(10) Pub. No.: US 2013/0267872 A1
(43) Pub. Date: Oct. 10, 2013

(54) HEALTH RISK MARKER MEASUREMENT DEVICE

(71) Applicant: Amrit Pal Singh Bhangoo, Boston, MA (US)

(72) Inventor: Amrit Pal Singh Bhangoo, Boston, MA (US)

(21) Appl. No.: 13/439,826

(22) Filed: Apr. 4, 2012

(51) Int. Cl. A61B 5/107

(52) U.S. Cl. 600/587

(57) ABSTRACT

A body part circumference measurement device is disclosed that incorporates a plurality of health risk markers which are customized to indicate the health risk levels for a single patient in different possible populations of patients. The body part used to generate the circumference measurement can be a patient's waist, neck, chest or any other body part whose circumference can be related, connected, correlated or associated with a particular health condition or risk. Each health risk marker can visually indicate or represent the severity or likelihood of an adverse health condition associated with a body part circumference which is equal to the position of the marker. The measuring device can be customized for children through the inclusion of age and/or gender specific markers. Visual appearance of the marker can be implemented in any way which distinguishes it from the surrounding material or area.

### BACKGROUND OF THE INVENTION

1. Field of the Invention

The present invention relates to a measurement device used to identify health risk factors in children and adults.

2. Background Art

Obesity has been linked to several serious medical conditions, such as high blood pressure, type-2 diabetes, heart disease, stroke, sleep apnea, fatty liver disease, high cholesterol, and many others. Therefore, an accurate assessment of a patient's risk for complications from obesity is necessary in order to get a complete picture of their health and determine whether they are at an increased risk for obesity and obesity related complications.

Doctors have utilized many methods to determine the patient's risk for obesity and obesity related complications, including general physical exams, medical history, diet, an assessment of a patient's Body Mass Index (“BMI”), body fat percentage, and/or skin fold thickness measurements using instruments such as skin fold calipers. The drawback to these methods is that they usually require multiple measurements of skin fold thickness or lengthy examinations and/or many tests and questions before an accurate diagnosis or risk assessment can be determined.

While those methods have been somewhat effective at diagnosing obesity or assessing the risks posed by obesity, body part circumference measurement has been shown to be a much simpler and more accurate method for obesity diagnosis and obesity related risk assessment. Among the more common circumference measurements used by doctors are waist circumference and neck circumference. Both waist and neck circumference can be used to quickly determine whether a patient is at risk for obesity and a variety of conditions, such as cardiovascular disease, metabolic syndrome, or high blood pressure.

While body part circumference can be readily obtained through a single measurement, doctors are still required to cross reference the measurement with guidelines concerning which circumference thresholds are considered at-risk for both men and women. Additionally, in children, these guidelines can vary depending on the age and gender of the child, resulting in additional time required to cross reference circumference measurements with the appropriate thresholds and adequately determine whether a particular child is at risk for adverse health effects. Furthermore, circumference measurements which are considered at-risk for obesity and obesity related conditions can also fluctuate between different ethnic groups. This has the effect of discouraging the use of body part circumference as a diagnostic and risk assessment method, despite evidence supporting it's effectiveness as a marker of obesity related health complications and simplicity of application.

With increasing numbers of patients at risk for serious health ailments due to rising obesity rates, particularly in children, there is a need for a method of obesity assessment which is accurate, easy to use, and allows for a quick diagnosis in diverse populations of children and adults without requiring the physician to consult outside reference material.

### BRIEF SUMMARY OF THE INVENTION

The present invention solves the problems of the prior art by providing a body part circumference measurement device that incorporates a plurality of health risk markers which are customized to indicate the health risk thresholds for different populations of patients. The body part used to generate the circumference measurement can be a patient's waist, neck, chest or any other body part whose circumference can be related, connected, correlated or associated with a particular health condition or risk.

In one non-limiting embodiment, the measuring device can be customized for children through the inclusion of age and/or gender specific markers. Each marker can indicate when a child of a specific age and/or gender is at risk for adverse health conditions based on the body part circumference measurement at the marker.

In another non-limiting embodiment, the measuring device can be customized for men and women, or for different ethnicities, and can contain a plurality of health risk markers for each sex and/or ethnic group. Each marker can visually indicate or represent the severity or likelihood of an adverse health condition associated with the body part circumference at that marker.

The health risk markers can be calibrated based on the average circumference for the particular body part for a particular population of patients, such as children of a particular age and gender, or men and/or women in a particular age group or of a particular ethnicity. Calibration of health risk markers can also be based on academic papers, statistical correlations, or scientific literature which links certain waist circumference sizes with a health risk for a particular population.

The health risk marker can indicate that the patient falls at, above or below a predetermined threshold relative to their peers in a particular population of patients. Marker can also reflect that a particular body part circumference meets or exceeds a particular level of risk for an adverse health condition.

The visual appearance of the marker can be implemented in any way which distinguishes it from the surrounding material or area, such as through the use of special materials, colors, fonts, labels, symbols, text, effects such as italics or bold, or any other visually perceptible technique or tactile technique which serves to bring the marker to the attention of the user.

### BRIEF DESCRIPTION OF THE OF THE SEVERAL VIEWS OF THE DRAWING(S)

FIG. 1 is an embodiment of the measuring device for children of different ages with the health risk markers for boys and girls of different ages on one side.

FIG. 2 is an embodiment of the measuring device for children of different ages with the health risk markers for boys on one side and girls on the opposite side.

FIG. 3 is the same measuring device as FIG. 2 with each of the markers for boys and girls grouped into two clearly labeled sets.

FIG. 4 is an embodiment of the measuring device for children of different ages with the health risk markers for boys on a border on one side and girls on a border on the opposite side.

FIG. 5 is an embodiment of the measuring device for adult men and women customized for different ethnic groups with the health risk markers for men on one side and the health risk markers for women on the opposite side.

### DETAILED DESCRIPTION OF THE INVENTION

The invention relates to a measurement device and method used to identify health risk factors in children and adults of different ethnic backgrounds. The measurement device can be in the form of a measuring tape which is marked with multiple Health Risk Markers (HRMs). Each of the HRMs can represent one of a plurality of different health risks as will be discussed below. Additionally, different embodiments of the measurement device can be customized to a plurality of different patient populations. Although the embodiments below deal specifically with waist circumference, the measurement device and associated HRMs can be customized to any body part circumference, such as the neck, or the chest, and is not limited to the embodiments below.

### I. Child & Adolescent Health Risk Measurement Device

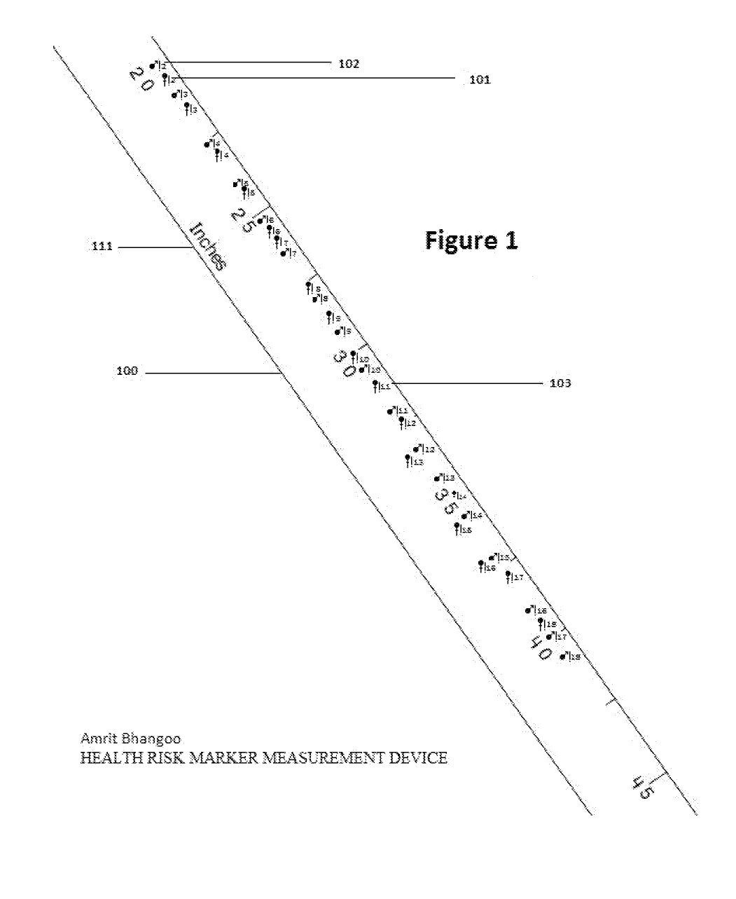

FIG. 1 illustrates an embodiment of the measurement device, 100, geared towards children and adolescents. Device, 100, includes units of measurement, 111, and a plurality of HRMs. The first type of HRM is represented by marker, 101. HRM, 101, represents a health risk for a female child whose waist measurement is equal to the distance marked by the HRM, 101, and who is of the age indicated by the HRM, 101. Similarly, HRM, 102, represents a health risk for a male child whose waist measurement is equal to the distance marked by HRM, 102, and who is of the age indicated by the HRM, 102. Each HRM in this embodiment includes a symbol representing the gender to which the HRM pertains and a number representing the age of the individual to which the HRM pertains. So, for example, HRM 103 indicates a health risk for an eleven year old girl whose waist circumference measures 31.4 inches.

The HRM placement in FIG. 1 is based on statistics indicating the 90th percentile of waist circumferences for male and female children from the ages of 2-18 (see Table 1 below). Children in the 90th percentile among their peers have been shown to be at an increased risk for many obesity related adverse health conditions. However, the invention is not limited to placing HRMs based on this measure, and may utilize a variety of methods, statistics and techniques to place HRMs. HRMs could be based on measures such as the 50th, 60th, 75th, or any other percentile of body part circumferences. HRMs can also indicate health risks associated with body part circumferences below a certain percentile, such as the 10th percentile, to help doctors diagnose or recognize ailments such as anorexia nervosa or other eating disorders. Additionally, each age and gender may include more than one HRM, indicating degrees of risk.

TABLE 1
90th percentile of waist circumferences for children

| Age | Boys | Girls |
|---|---|---|
| 2 | 20.0 inches | 20.6 inches |
| 3 | 21.3 inches | 21.8 inches |
| 4 | 22.6 inches | 23.0 inches |
| 5 | 24.0 inches | 24.2 inches |
| 6 | 25.3 inches | 25.4 inches |
| 7 | 26.7 inches | 26.6 inches |
| 8 | 28.0 inches | 27.8 inches |
| 9 | 29.4 inches | 29.0 inches |
| 10 | 30.7 inches | 30.2 inches |
| 11 | 32.1 inches | 31.4 inches |
| 12 | 33.4 inches | 32.6 inches |
| 13 | 34.7 inches | 33.8 inches |
| 14 | 36.0 inches | 35.0 inches |
| 15 | 37.4 inches | 36.2 inches |
| 16 | 38.7 inches | 37.4 inches |
| 17 | 40.1 inches | 38.6 inches |
| 18 | 41.4 inches | 39.8 inches |

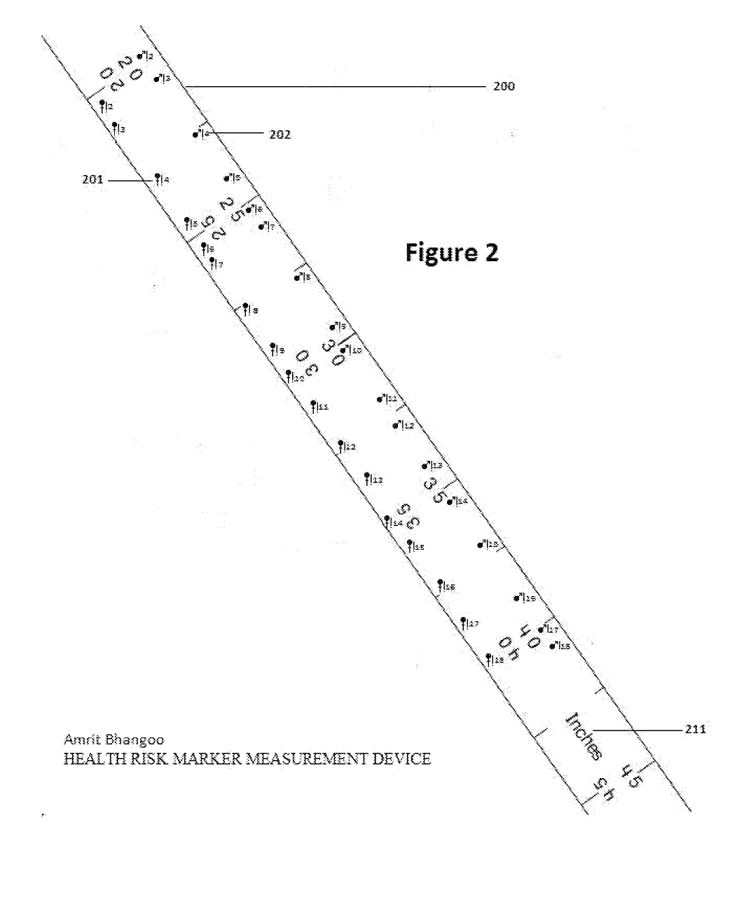

FIG. 2 illustrates an embodiment of the measurement device, 200, geared towards children and adolescents. The device, 200, is similar in many respects to the measurement device, 100, of FIG. 1, but has the HRMs for girls, represented by 201, and boys, represented by 202, on opposite sides of the device. This makes the HRMs on the device much easier to read and use, since the doctor can just change the orientation of the device depending on the gender of the patient and easily determine whether their waist circumference puts them at risk.

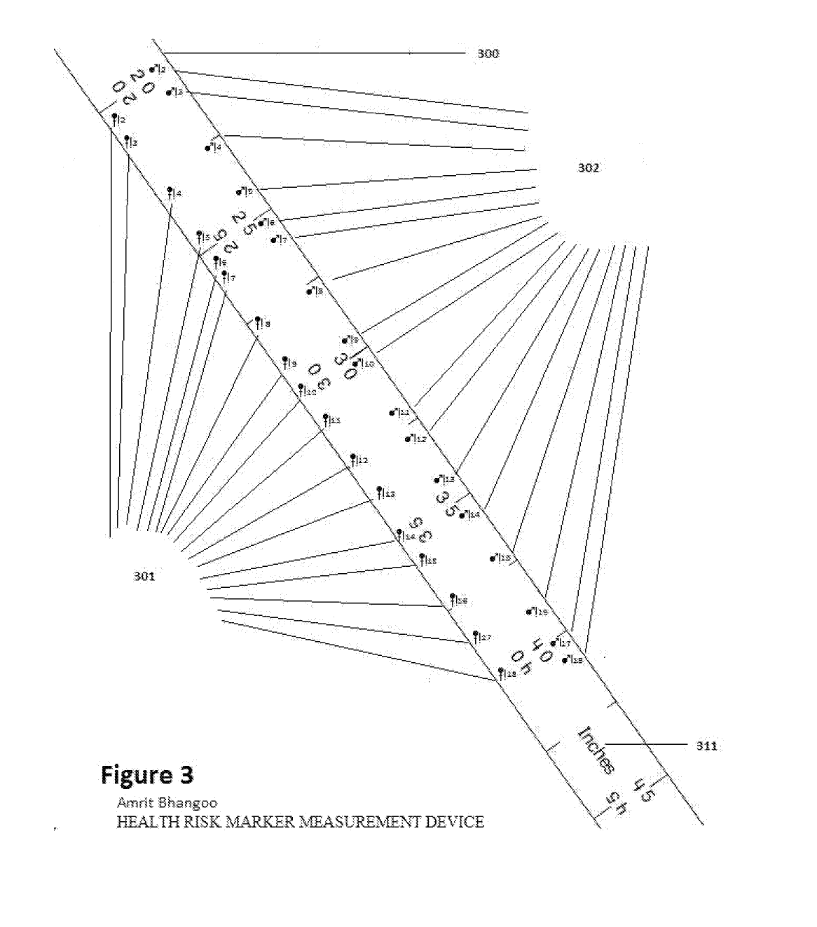

FIG. 3 shows the measurement device of FIG. 2 with each of the HRMs clearly labeled. HRMs that form the set, 301, are the risk markers for girls between the ages of 2-18. The HRMs that form the set, 302, are the risk markers for boys between the ages of 2-18.

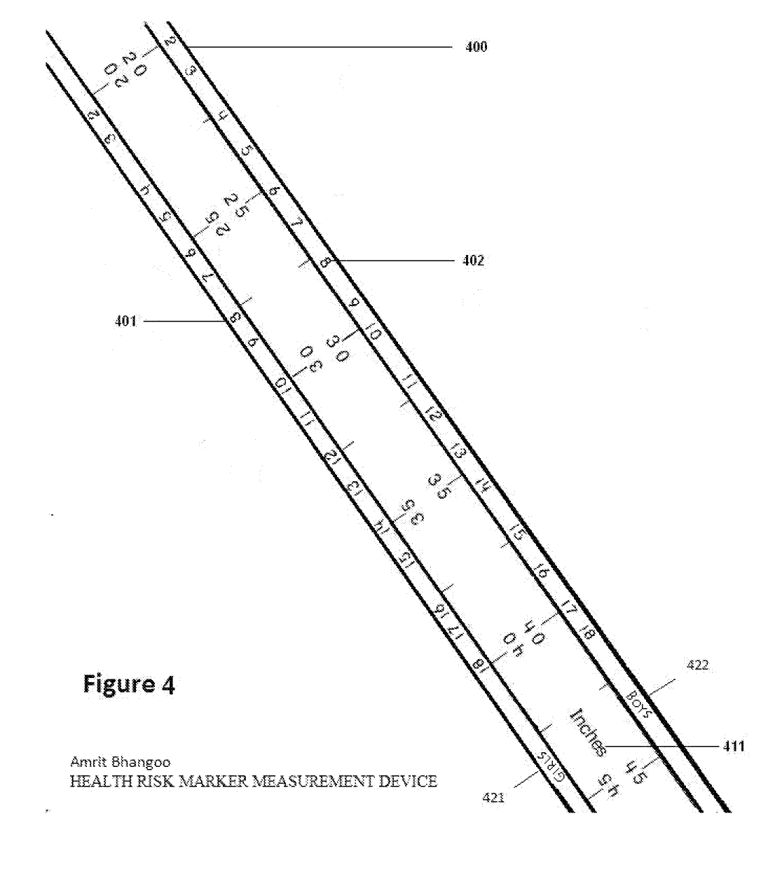

FIG. 4 shows another embodiment of the measurement device, 400, which is also intended for children and adolescents. In this embodiment, the HRMs for girls, 401, and the HRMs for boys, 402, are also on opposite sides of the tape, but are contained on two borders which form the outer edge of the tape. The HRMs in this embodiment are shown as just listing the age number at which the specified circumference is a health risk, but other embodiments could include other symbols and markers as discussed below. To avoid crowding and for the sake of clarity, the symbols for boys and girls in this embodiment are replaced by indicators for a girls side, 421, and a boys side, 422, at a single location on the tape.

### II. Risk Measurement Device Among Different Ethnic Groups

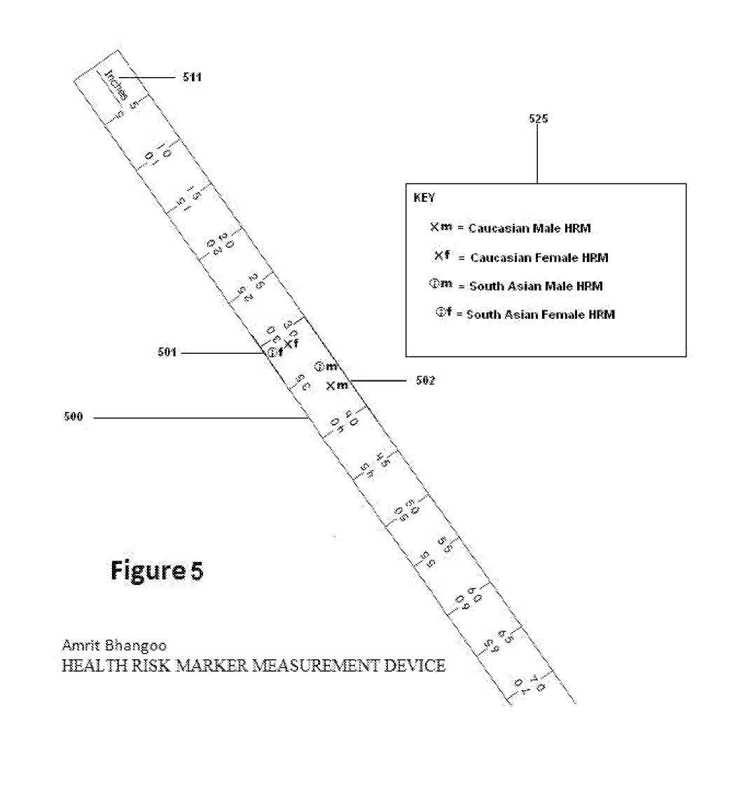

FIG. 5 illustrates an embodiment of the measurement device, 500, geared to adults from different ethnic backgrounds. Measurement device, 500, is marked with units of measurement, 511, shown in the figure as inches. Two sets of HRMs are shown on device, 500. The first set of HRMs, comprised of markers, 501, indicate a health risk for adult women whose waist circumference is equal to the distance indicated by the marker, and are presented on one side of the tape. Markers, 502, indicate health risks for men, and are located on the opposite side of the tape.

Additionally, FIG. 5 includes different markers for two different ethnic groups. The key, 525, illustrates that the two HRMs for men and the two HRMs for women represent South Asian males, Caucasian males, South Asian Females, and Caucasian Females. These ethnic groups are provided for illustration only, and the measurement device, 500, could easily have any number of HRMs corresponding to males and females in a plurality of different ethnic groups. As stated earlier, the device and HRMs are not limited to waist circumference, and can utilize any body part of the patient which can be correlated to a health condition or risk.

### III. Risk Measurement Device Among Different Ethnic and Age Groups

The measurement device may also be tailored to males and females in plurality of ethnic and age groups. In such an embodiment, each HRM would indicate obesity or an obesity related health risk for a person in a population demographic, said population demographic comprising a specific ethnic group, age group, and gender.

### IV. Measurement Device Construction

The measurement device disclosed in the application and shown in FIGS. 1-5 can be comprised of any suitable material such as plastics, cloths, leathers, fabrics, fiber glass or metals. Additionally, measurement device, can be comprised of a disposable or recyclable material such as paper. Such a construction is preferable in a clinical setting where doctors must sanitize or discard any materials which make contact with a patient's skin. Measurement device can be implemented as a tape or a retractable tape as is well known in the art. Measurement device can also be implemented on a flexible electronic display using e-ink or a bendable light emitting diode. In such a case, HRMs on the display could be dynamically or automatically updated with current information reflecting at-risk demographic information in patient populations.

### V. Units of Measurement

The units of measurement used for measurement device are shown as inches in FIGS. 1-5 but can be expressed in centimeters, millimeters, meters, or any other unit of distance measurement, or by using multiple units of measurement or any combination of units.

### VI. Health Risk Marker Appearance

HRMs are shown as a set of symbols in FIGS. 1-5 but the visual appearance of the marker can be implemented in any way which distinguishes it from the surrounding material or area, such as through the use of special materials, textures, colors, fonts, labels, symbols, text, effects such as italics or bold, or any other visually perceptible technique which serves to bring the marker to the attention of the user. Furthermore, the difference between male and female, or age based HRMs does not have have to be a symbol, but can be an orientation, placement, special material, texture, color, font, label, symbol, text, effects such as italics or bold, or any other visually perceptible technique.

### VII. Health Risk Marker Quantity, Calibration, and Placement

The number of HRMs for a particular patient, group, or population is not limited to one, and can be adjusted to suit the needs of the user. Multiple HRMs can be used for each age or gender combination, indicating multiple levels of risk or different probabilities of adverse health effects. The health effects are not limited to the diseases or conditions set forth in this disclosure and can include any health ailment which can be related, correlated, or linked to waist circumference. Calibration of health risk markers can also be based on academic papers, statistical correlations, or scientific literature which links certain waist circumference sizes with a health risk for a particular population. Additionally, measurement device can be customized for groups other those based on gender, such as for example, ethnicity.

### VIII. Body Part Used for Measurement

The body part part of the patient which is measured by the device and to which the HRMs correspond can be any body part for which the circumference, length, or other dimension can be linked to a health condition or risk. This can include the patient's waist, neck, chest, arm, leg, or any other part which is found to indicate any type of health risk.

### IX. Obesity Related Adverse Health Conditions

This disclosure makes reference to obesity related adverse health conditions, adverse health conditions, health risks, and/or health complications that can be assessed using the measurement device disclose herein. Such health conditions include, but are not limited to: obesity, high blood pressure, type-2 diabetes, heart disease, stroke, sleep apnea, fatty liver disease, high cholesterol, and/or any other condition which has been or can be correlated to obesity and/or a specific body part measurement.

1. A measurement device incorporating health related markers for diverse populations of users, comprising:
a plurality of age groups represented on the measurement device; and
a plurality of health related markers disposed on the measurement device;
wherein each health related marker is associated with an age group out of the plurality of age groups and a gender out of one or more genders; and
wherein each health related marker is disposed at a position on the measurement device which corresponds to a health risk for a person who belongs to the age group and the gender associated with the marker, and who possesses a body part measurement which is equal to the length of the measurement device at the location of the health related marker.

2. The measurement device of claim 1, wherein
the measurement device comprises a measuring tape or a measuring belt;
the body part measurement comprises a circumference measurement; and
each health related marker is disposed at a position on the measurement device which corresponds to a health risk for a person who belongs to the age group and the gender associated with the marker, and who possesses a body part circumference which is equal to the length of the measurement device at the location of the health related marker.

3. The measurement device of claim 2, wherein the measurement device comprises a measuring tape and wherein:
the location of each health related marker on the measuring tape is a predetermined distance from the start of the tape; and
the predetermined distance is equal to the body part circumference of a subset of a population segment defined by the age group and gender associated with the health risk marker.

4. The measurement device of claim 3, wherein the subset comprises the subset of the population segment who have a body part circumference corresponding to a given percentile relative to their peers in the population segment.

5. The measurement device of claim 4, wherein the health related marker comprises a visual or tactile indication of the age group and the gender to which it pertains.

6. The measurement device of claim 4, wherein the given percentile comprises a percentile between the fiftieth and hundredth percentiles, inclusive.

7. The measurement device of claim 6, wherein the health risk comprises at least one of obesity, high blood pressure, diabetes, heart disease, stroke, sleep apnea, fatty liver disease, high cholesterol, cardiovascular disease, and metabolic syndrome.

8. The measurement device of claim 4, wherein:
multiple health related markers are associated with the population segment; and
each of the multiple health related markers associated with the population segment correspond to a different subset of the population segment.

9. The measurement device of claim 2, wherein the body part circumference is selected from the group consisting of: chest circumference, waist circumference, and neck circumference.

10. The measurement device of claim 2, wherein the measurement device comprises at least one of cloth, clothing, fabric, leather, metal, paper, and plastic.

11. The measurement device of claim 2, wherein:
the measurement device further comprises a representation of units of measurement; and
the units of measurement comprise at least one of inches, meters, millimeters, centimeters, and yards.

12. The measurement device of claim 2, wherein the health related markers comprise at least one of italicized text, bold text, textured materials, numbers, colors, fonts, labels, symbols, and text.

13. The measurement device of claim 2, wherein:
the location of each health related marker on the measuring tape is a predetermined distance from the start of the tape; and
the predetermined distance is equal to a body part circumference which has been correlated with an adverse health condition for a population segment defined by the age group and the gender associated with the health related marker.

14. A measurement device incorporating health related markers for diverse populations of users, comprising:
a plurality of ethnic groups represented on the measurement device; and
a plurality of health related markers disposed on the measurement device;
wherein each health related marker is associated with an age group out of the plurality of age groups and a gender out of one or more genders; and
wherein each health related marker is disposed at a position on the measurement device which corresponds to a health risk for a person who belongs to the ethnic group and the gender associated with the marker, and who possesses a body part circumference which is equal to the length of the measurement device at the location of the health related marker.

15. The measurement device of claim 14, wherein:
the location of each health related marker on the measuring tape is a predetermined distance from the start of the tape; and
the predetermined distance is equal to the body part circumference of a subset of a population segment defined by the ethnic group and gender associated with the health risk marker.

16. The measurement device of claim 15, wherein the subset comprises the subset of the population segment who have a body part circumference corresponding to a given percentile relative to their peers in the population segment.

17. The measurement device of claim 16, wherein the health related marker comprises a visual or tactile indication of the ethnic group and gender to which it pertains.

18. (canceled)

19. The measurement device of claim 14, wherein:
the location of each health related marker on the measuring tape is a predetermined distance from the start of the tape; and
the predetermined distance is equal to a body part circumference which has been correlated with an adverse health condition for a population segment defined by the ethnic group and gender.

20. (canceled)

21. A measurement device incorporating health related markers, comprising:
a plurality of age groups represented on the measurement device; and
a plurality of health related markers disposed on the measurement device;
wherein each health related marker is associated with an age group out of the plurality of age groups and a gender out of one or more genders; and
wherein each health related marker is disposed at a position on the measurement device which corresponds to a health risk for an animal who belongs to the age group and the gender associated with the marker, and who possesses a body part measurement which is equal to the length of the measurement device at the location of the health related marker.

22. The measurement device of claim 21, wherein the animal is a person.
